(19) United States
(12) Patent Application Publication
(10) Pub. No.: US 2008/0064701 A1
(43) Pub. Date: Mar. 13, 2008

(54) Anti-diabetic combinations

(76) Inventor: Ramesh Sesha, West Windsor, NJ (US)

Correspondence Address: Ramesh Sesha, 9113 Taylor Court, West Windsor, NJ, 08550, US

(21) Appl. No.: 11/789,080

(22) Filed: Apr. 24, 2007

(51) Int. Cl. A61K 31/50; A61K 31/155; A61P 3/10

(52) U.S. Cl. 514/249; 514/635

(57) ABSTRACT

The invention discloses a method of administering an anti-diabetic combination comprising a DPP inhibitor and a slow release biguanide to a mammal in need of thereof. This invention further discloses anti-diabetic combination comprising a DPP inhibitor and a slow release biguanide for treating diabetes.

### BACKGROUND OF THE INVENTION

Diabetes mellitus of type II is a progressive metabolic disorder with diverse pathologic manifestations and is often associated with lipid metabolism and glycometabolic disorders. The long-term effects of diabetes result from its vascular complications; the microvascular complications of retinopathy, neuropathy and nephropathy and the macrovascular complications of cardiovascular, cerebrovascular and peripheral vascular diseases. Initially, diet and exercise is the mainstay of treatment of type II diabetes. However, these are followed by administration of oral hypoglycemic agents. Current drugs used for managing type II diabetes and its precursor syndromes such as insulin resistance include classes of compounds¹. The main classes of anti-diabetic drugs are outlined below
A. Biguanides:
   This class of drugs includes metformin, phenformin, buformin and the like with metformin is widely used in immediate release and in extended release forms. Biguanides principally help in the control of blood glucose by inhibiting hepatic glucose production, reducing intestinal absorption of glucose and enhancing peripheral glucose uptake. Biguanides, especially metformin, lowers both basal and post-prandial plasma glucose and thus improves tolerance of glucose in patients. Metformin exerts normoglycemic action with reduced risk of lactic acidosis and is also known to lower blood triglyceride levels. It is therefore a preferred mode of therapy among biguanides. Metformin is widely viewed as the initial drug of choice for the treatment of T2DM, owing to its 30-year track record, efficacy, safety and low cost.
B. Sulfonylurea:
   The sulfonylureas include tolbutamide, tolazamide, chlorpropamide and the more recent glipizide, glyburide and glimepiride. Sulfonylureas, represented principally by glipizide, glimiperide, glyburide, glibomuride, glisoxepide, gliclazide acetohexamide, chlorpropamide, tolazamide, and tolbutamide, among others, help in controlling or managing NIDDM by stimulating the release of endogenous insulin from the beta cells of the pancreas^{1 3}.
C. Thiazolidinediones:
   This class includes Troglitazone, Pioglitazone, Rosiglitazone, Ciglitazone, Isaglitazone, Darglitazone, zorglitazone, Englitazone, Balaglitazone and the like. Glitazones, represented principally by the class of glitazones including, for example, rosiglitazone, troglitazone and pioglitazone, among others, act by increasing the sensitivity of insulin receptors in the body and decreasing peripheral insulin resistance. Glitazones, preferably pioglitazone, stimulate adipogenesis and reduce plasma triglyceride and free fatty acid concentrations. These enhance insulin action at the cellular level but do not stimulate insulin release, nor do they mimic its action^{1,3}
D. α-Glycosidase Inhibitors:
   Alpha-glucosidase inhibitors, acarbose (AK-er-bose) and miglitol (MIG-leh-tall). Both medicines block the enzymes that digest the starches eaten. This action causes a slower and lower rise of blood glucose through the day, but mainly right after meals. Neither acarbose nor miglitol causes hypoglycemia when it is the only diabetes medicine. One of the biggest drawbacks to the alpha-glucosidase inhibitors is their side effects. Because they affect carbohydrate absorption in the small intestine, they can cause bloating, nausea, diarrhea, and flatulence. This class includes acarbose, miglitol, voglibose, emiglitate, and the like.
E. Meglitinides:
   This class of drugs includes Repaglinide, Nateglinide. Non-sulfonylureal insulin secretagogues, also known as the “meglitinides,” lower blood sugar levels by stimulating the release of insulin from the pancreas in response to glucose (from food). Insulin is required to move sugar from the bloodstream into the cells of the body where it can be used as energy. To work best, meglitinides should be taken immediately before a meal.
F. DPP4 Inhibitors:
   Dipeptidyl peptidase (DPP4) inhibitors, that include Sitagliptin, Vildagliptin and Saxagliptin, are a new class of drugs that inhibit the proteolytic activity of dipeptidyl peptidase-4, thereby potentiating the action of endogenous glucoregulatory peptides, known as incretins. They are orally-bioavailable selective DPP4 inhibitors that were discovered through the optimization of a class of -amino-acid-derived DPP4 inhibitors. It lowers DPP4 activity in a sustained manner following once daily administration, preserves the circulating levels of intact GIP and GLP1 following meals in both acute and chronic studies and reduces blood glucose levels without significant increases in hypoglycaemia².

DPP4 inhibitors, biguanides, glitazones and sulfonylureas are commercially available in the form of tablets of the individual drugs, either as immediate release (IR) formulations or in some cases controlled release (CR) formulations, to be administered orally to patients in need thereof, in protocols calling for the single administration of the individual ingredient.

However, many physicians now advocate initiating therapy of T2DM with at least two drugs to obviate the monotherapy failure that accompanies prolonged metformin use in the majority of treated patients^{1 3 & 4}. Metformin monotherapy is used as a first line treatment in diabetic patients but may be supplemented with other drugs when the secondary failure of the therapy sets in. The addition of a DPP inhibitor, glitazones and sulfonylurea to the concurrent treatment provides a balance of stimulated release of insulin while ameliorating insulin resistance and thus provides an optimal level of glycemic control unattainable by either medication alone.

Insulin resistance and reduced insulin secretion are the two fundamental abnormalities in type 2 diabetic patients. Therefore, reducing insulin resistance or increasing insulin sensitivity and augmenting insulin secretion from beta cells of pancreas are the two major treatment approaches. The tissues most commonly resistant to actions of insulin are liver, skeletal muscles, and adipose tissues. Therefore, combination treatment strategies directed towards improving the insulin sensitivity of these major tissues help in overall enhancement of insulin sensitivity.

A brief logical profile for such combinations based on the pharmacological mechanism of action of the individual classes of drugs is listed below:
a. Metformin and Glyburide (or Glibenclamide)
b. Metformin and Glipizide
c. Metformin and Pioglitazone
d. Metformin and Rosiglitazone
e. Glimepiride and Rosiglitazone

The safety and efficacy of a DPP inhibitor sitagliptin as a monotherapy and in combination with existing anti-diabetic agents was assessed in four randomized double-blind placebo-controlled clinical trials that involved more than 2,000 patients with T2DM^{6, 7, 8, 9, 10}. Several measurements relevant to glycemic control were evaluated, including the mean change from baseline in glycated hemoglobin (HbA1C) levels—an indicator of average blood-sugar levels for the past 3-4 months. Sitagliptin as a monotherapy, at doses of either 100 or 200 mg daily significantly reduced HbA1C, with few adverse events, and no significant increase in hypoglycemia ^{7,8}. The extent of HbA1C reduction was proportional to the starting HbA1C, and no significant weight gain was observed in 24-week monotherapy studies. Sitagliptin reduced both fasting and postprandial glycaemia, in association with improvements in the proinsulin/insulin ratio and homeostatic model assessment of -cell function (HOMA-B)⁸. For patients who did not achieve adequate glycemic control on at least 1,500 mg per day of metformin (mean HbA1C of 8%), the addition of sitagliptin 100 mg daily resulted in 47% of patients achieving a HbA1C of <7%, compared with 18.3% of placebo-treated subjects⁹. The mean placebo-subtracted reduction in HbA1C was 0.65%, and sitagliptin therapy was also associated with significant reductions in fasting glucose and increases in parameters of -cell function. Sitagliptin has also been shown to be effective when combined with metformin as initial therapy for T2DM. In 24-week studies of sitagliptin as an add-on therapy for patients not achieving adequate glycemic control (mean HbA1C 8.1%) on pioglitazone (30 or 45 mg daily), sitagliptin at a dose of 100 mg daily produced a mean HbA1C reduction of 0.7%, and significantly greater numbers of patients achieved a HbA1C of <7% on sitagliptin relative to pioglitazone alone (45.4 versus 23%, respectively)¹⁰. Sitagliptin therapy was not associated with increased rates of hypoglycemia or weight gain relative to patients treated with pioglitazone alone.

Pharmaceutical dosage forms containing combinations of anti-diabetic drugs have been proposed in the art. For example, EPO 0 749 751 (which is incorporated herein by reference) teaches pharmaceutical compositions comprising an insulin sensitivity enhancer, which could be a thiazolidinedione compound, in combination with other anti-diabetics. More specifically, EPO 0 749 751 teaches that the preferred insulin sensitivity enhancer is pioglitazone, which can be combined with other anti-diabetics such as metformin, phenformin or buformin, and further that these drugs can be associated (mixed and/or coated) with conventional excipients to provide taste masking or sustained release behavior. Another example of a combination of antihyperglycemic drugs and thiazolidinedione derivatives is U.S. Pat. No. 6,011,049, which is incorporated herein by reference. This patent teaches a single pharmaceutical composition that contains pioglitazone or trolitazone and metformin in slow release forms such as osmotic pumps or skin patches. Other combinations of antihyperglycemic drugs and thiazolidinedione derivatives can be found in U.S. Pat. Nos. 6,524,621; 6,475,521; 6,451,342 and 6,153,632 and PCT patent applications WO 01/3594 and WO 01/3594, which are incorporated herein by reference. U.S. Pat. No. 7,125,873 describes pharmaceutical composition comprising a DPP4 inhibitor like Sitagliptin with other anti-diabetic drugs like biguanide, PPAR agonists

However, some of these combinations, for example; Actoplus Met (Pioglitazone+Metformin) and Duetact (Pioglitazone+Glimepiride) pose medical risks like fracture of bones in certain sections of patient. Hence efforts are needed to find better and safer anti-diabetic combinations. The present invention was undertaken with such an objective. Although the prior art teaches pharmaceutical dosage formulations that contain combination drugs, the present invention provides numerous benefits over the prior art teaching. The present invention surprisingly provide an anti-diabetic combination comprising a DPP4 inhibitor and a slow release biguanide that is superior, to either a combination of two immediate release drugs or monotherapy, for treating diabetes. Further the present invention to provide a method of administering the combination of a DPP4 inhibitor and a slow release biguanide that provide the following advantages
1. The combination targets the different major pathological processes, insulin resistance and potentiation of glucose-dependent insulin secretion using a combination of drugs
2. The therapeutic objective is achieved with the combination of a DPP4 inhibitor, and a slow release biguanide.
3. Increased insulin sensitivity and secretion due to synergistic actions of the combination drug.
4. Therapeutic actions of combination are enhanced due to their release over a period of time.
5. Better glycemic control.
6. Reduced incidence of side effects due reduced dosage requirements of individual drugs.
7. Improved compliance because the combination is provided as a fixed dose

It is an object of the present invention to provide an anti-diabetic combination comprising a DPP4 inhibitor and a slow release biguanide

It is further an object of the present invention to provide a method of administering an anti-diabetic combination comprising a DPP4 inhibitor and a slow release biguanide

It is another object of the present invention to provide an anti-diabetic combination kit comprising a DPP4 inhibitor and a slow release biguanide

It is yet another object of present invention to provide a pharmaceutical composition comprising a DPP4 inhibitor and a slow release biguanide.

It is an additional object of the present invention to provide an anti-diabetic combination comprising a DPP4 inhibitor and a slow release biguanide wherein the peak plasma levels of a DPP inhibitor is approximately 1-4 hours after dosing.

Further it an object of the present invention to provide an anti-diabetic combination comprising a DPP4 inhibitor and a slow release biguanide wherein not less than 85% of the total amount of the DPP4 inhibitor is released from the dosage form within 120 minutes or less.

### BRIEF DESCRIPTION OF THE INVENTION

One object of the present invention is to provide methods, which can effectively be used in the treatment of diabetes and diabetes related diseases wherein the methods comprise administration of an effective amount of a DPP4 inhibitor, and administration of an effective amount of a slow release biguanide to a patient in need thereof.

Another object of the invention is to provide methods for increasing the number of beta-cells in a patient, increasing the size of beta-cells in a patient or stimulating beta-cell proliferation in a patient in need thereof, which method comprises administration of an effective amount of a DPP4 inhibitor and administration of an effective amount of a slow release biguanide to a patient in need thereof.

The two anti-diabetic drugs, i.e. the DPP4 inhibitor and a slow release biguanide, may be co- administered or they may be administered separately as two medicaments. Furthermore, the first drug may be administered in a regimen, which additionally comprises treatment with the second drug or third drug or the combination of first drug.

In one embodiment of the invention, the DPP4 inhibitor is Sitagliptin, or its respective pharmaceutically equivalent derivative and the slow release biguanide is metformin or its pharmaceutically equivalent derivative.

In yet another embodiment of the invention the DPP4 inhibitor and a slow release the biguanide are administered in suboptimal dosages.

In yet another embodiment of the invention the DPP4 inhibitor and a slow release the biguanide are administered in amounts and for a sufficient time to produce a synergistic effect.

### DETAILED DESCRIPTION OF THE INVENTION

The following is a detailed definition of the terms used in the specification.

The term, “biguanide” as used in this specification, refers to drugs that are useful in controlling or managing non-insulin-dependent diabetes mellitus (NIDDM). They include the biguanides such as metformin, phenformin or buformin or the like and pharmaceutically acceptable salts, isomers or derivatives thereof.

The term “Sulfonylurea” as used in this specification refers principally drugs like glipizide, glimiperide, glyburide, glibomuride, glisoxepide, gliclazide acetohexamide, chlorpropamide, tolazamide, and tolbutamide, among others, that help in controlling or managing NIDDM by stimulating the release of endogenous insulin from the beta cells of the pancreas^{1 3}

The term “DPP4 Inhibitor” as used in this specification refers to drugs that are useful for controlling or managing NIDDM. These include, but are not limited to, Sitagliptin, Saxagliptin, Vildagliptin and all other molecular entities such as SYR 522 (pyrimidine derivatives), PHX1149, GRC-8200 (tricyclic derivatives), SSR162369 (biocyclic 8-pyrrolidinoxanthine) derivatives that inhibit DPP4 protease in a mammal²

The term “diabetes and diabetes related diseases” as employed herein refers to the disease selected from the group consisting of type 1 diabetes, type 2 diabetes, hyperglycemia, type 1.5 diabetes, latent autoimmune diabetes in adults, maturity onset diabetes, beta-cell apoptosis, hemochromatosis induced diabetes, impaired glucose tolerance, metabolic syndrome X, insulin resistance, cystic fibrosis related diabetes, polycystic ovarian syndrome, gestational diabetes, obesity, dyslipidemia, diabetic dyslipidemia, hyperlipidemia, hypertriglyceridemia, hyperlipoproteinemia, hypercholesterolemia, hypertension, essential hypertension, acute hypertensive emergency, arteriosclerosis, atherosclerosis, intermittent claudication (atherosclerosis oblilterens), cardiovascular disease, cardiomyopathy, cardiac hypertrophy, left ventricular hypertrophy, coronary artery disease, early coronary artery disease, heart insufficiency, exercise tolerance, chronic heart failure, mild chronic heart failure, arrhythmia, cardiac dysrythmia, syncopy, heart attack, myocardial infarction, Q-wave myocardial infarction, stroke, acute coronary syndrome, angina pectoris, unstable angina, cardiac bypass reocclusion, diastolic dysfunction, systolic dysfunction, non-Q-wave cardiac necrosis, catabolic changes after surgery, acute pancreatitis, irritable bowel syndrome, diabetic retinopathy, background retinopathy, preproliferative retinopathy, proliferative retinopathy, macular edema, cataracts, nephropathy, diabetic nephropathy, microalbuminuria, macroalbuminuria, neuropathy, diabetic neuropathy, distal symmetrical sensorimotor polyneuropathy, and diabetic autonomic neuropathy.

The term “co-administration” as used herein means administration of the two compounds to the patient within a period of one month. The term includes separate administration of two medicaments each containing one of the compounds as well as simultaneous administration whether or not the two compounds are combined in one formulation or whether they are in two separate formulations.

The term “effective amount” as used herein means a dosage which is sufficient in order for the treatment of the patient to be effective compared with no treatment.

The term “medicament” as used herein means a pharmaceutical composition suitable for administration of the pharmaceutically active compound to a patient.

The term “suboptimal dosage” us used herein means a dosage which is below the optimal dosage for that compound when used in single-compound therapy.

The term “additive effect” as used herein means the effect resulting from the sum of the effects obtained from the individual compounds.

The term “synergistic effect” as used herein means an effect which is greater than the additive effect which results from the sum of the effects of the two individual compounds.

The term “treatment of a disease” as used herein means the management and care of a patient having developed the disease, condition or disorder. The purpose of treatment is to combat the disease, condition or disorder. Treatment includes the administration of the active compounds to eliminate or control the disease, condition or disorder as well as to alleviate the symptoms or complications associated with the disease, condition or disorder.

The term “prevention of a disease” as used herein is defined as the management and care of an individual at risk of developing the disease prior to the clinical onset of the disease. The purpose of prevention is to combat the development of the disease, condition or disorder, and includes the administration of the active compounds to prevent or delay the onset of the symptoms or complications and to prevent or delay the development of related diseases, conditions or disorders.

The term “extended release material” as present in the inner solid particulate phase and the outer solid continuous phase refers to one or more hydrophilic polymers and/or one or more hydrophobic polymers and/or one or more other type hydrophobic materials, such as, for example, one or more waxes, fatty alcohols and/or fatty acid esters. The “extended release material” present in the inner solid particulate phase may be the same as or different from the “extended release material” present in the outer solid continuous phase.

The term “slow-release” here applies to any release from a formulation that is other than an immediate release wherein the release of the active ingredient is slow in nature. This includes various terms used interchangeably in the pharmaceutical context like extended release, delayed release, sustained release, controlled release, timed release, specific release, targeted release etc

The term “candidate for sustained release” encompasses all the characteristics of a drug which make it a candidate for formulating it into an extended release fashion like a short elimination half life and consequent dosing of more than once a day, a single dose product given in an extended fashion to achieve better clinical results and avoid side effects associated with an immediate release etc

The term “binding agent” as used in this specification, refers to any conventionally known pharmaceutically acceptable binder such as polyvinyl pyrrolidone, hydroxypropyl cellulose, hydroxyethyl cellulose, hydroxypropyl methylcellulose, ethylcellulose, polymethacrylate, polyvinylalcohol, waxes and the like. Mixtures of the aforementioned binding agents may also be used. The preferred binding agents are water soluble materials such as polyvinyl pyrrolidone having a weight average molecular weight of 25,000 to 3,000,000. The binding agent may comprise approximately about 0 to about 40% of the total weight of the core and preferably about 3% to about 15% of the total weight of the core. In one embodiment, the use of a binding agent in the core is optional.

The term “pharmaceutically acceptable derivative” means various pharmaceutical equivalent isomers, enantiomers, complexes, salts, hydrates, polymorphs, esters etc of duloxetine

The term “therapeutically effective amount” means an amount that elicits a biological response in a mammal including the suboptimal amount

The term “gelling or swelling polymer” as used in this specification, refers to polymers that gel, swell or expand in the presence of water or biological fluids. Representative examples of gelling or swelling polymers are high molecular weight hydroxypropyl methylcellulose (such as METHOCEL.®. K100M, which is commercially available from Dow Chemical) and high molecular weight polyethylene oxides (such as POLYOX WSR 301, WSR 303 or WSR COAGULANT). Other gelling or swelling polymers are described in U.S. Pat. No. 4,522,625 (which is incorporated herein by reference).

The term “seal coat” as defined in this invention is a coating that does not contain an active pharmaceutical ingredient and that rapidly disperses or dissolves in water.

A pore forming is preferably a water-soluble material such as sodium chloride, potassium chloride, sucrose, sorbitol, mannitol, polyethylene glycols (PEG), propylene glycol, hydroxypropyl cellulose, hydroxypropyl methycellulose, hydroxypropyl methycellulose phthalate, cellulose acetate phthalate, polyvinyl alcohols, methacrylic acid copolymers, poloxamers (such as LUTROL F68, LUTROL F127, LUTROL F108 which are commercially available from BASF) and mixtures thereof.

The term “Hydrophilic polymers” as used in this specification include, but are not limited, to hydroxypropylmethylcellulose, hydroxypropylcellulose, sodium carboxymethylcellulose, carboxymethylcellulose calcium, ammonium alginate, sodium alginate, potassium alginate, calcium alginate, propylene glycol alginate, alginic acid, polyvinyl alcohol, povidone, carbomer, potassium pectate, potassium pectinate, etc

The term “Hydrophobic polymers” as used in this specification include, but are not limited, to ethyl cellulose, hydroxyethylcellulose, ammonio methacrylate copolymer (Eudragit RL.™. or Eudragit RS.™.), methacrylic acid copolymers (Eudragit L.™. or Eudragit S.™.), methacrylic acid-acrylic acid ethyl ester copolymer (Eudragit L 100-5.™.), methacrylic acid esters neutral copolymer (Eudragit NE 30D.™.), dimethylaminoethylmethacrylate-methacrylic acid esters copolymer (Eudragit E 100.™.), vinyl methyl ether/malefic anhydride copolymers, their salts and esters (Gantrez.™.) etc.

Other hydrophobic materials which may be employed in the inner solid particulate phase and/or outer solid continuous phase include, but are not limited, to waxes such as beeswax, carnauba wax, microcrystalline wax, and ozokerite; fatty alcohols such as cetostearyl alcohol, stearyl alcohol; cetyl alcohol myristyl alcohol etc; and fatty acid esters such as glyceryl monostearate, glycerol monooleate, acetylated monoglycerides, tristearin, tripalmitin, cetyl esters wax, glyceryl palmitostearate, glyceryl behenate, hydrogenated castor oil, etc.

The present invention provides an anti-diabetic combination for the treatment of diabetes and diabetes related diseases. According to this invention, a DPP4 inhibitor is used in combination with a slow release biguanide, to treat diabetes and diabetes related diseases and to improve glycemic control in patients in need of treatment.

According to this invention, the compounds can be employed individually, or can be combined in a single formulation, for example as a tablet, capsule, syrup, solution, as well as controlled release formulations. In a preferred embodiment, the DPP4 inhibitor and a slow release biguanide are formulated individually and administered in the same manner that each is normally used clinically.

Furthermore, the first drug may be administered in a regimen, which additionally comprises treatment with the second drug. Hence, according to the present invention the only provision is that there must be overlapping periods of treatment with the DPP4 inhibitor with slow release biguanide.

Typical combinations to be employed according to this invention thus include sitagliptin plus a slow release metformin. Another typical and preferred combination is vildagliptin plus a slow release metformin. Still yet another preferred combination is saxagliptin plus slow release metformin. These combinations produce better than expected therapeutic benefit in the treatment of diabetes and diabetes related diseases.

The dosage of each agent that needs to administered is determined the attending physician who would consider the severity of the disease, the frequency of administration, the particular agents and combinations utilized, and other factors routinely considered in a diabetic practice. Typically the DPP inhibitors will normally be administered at doses from about 50 mg to about 200 mg per day, and more typically from about 100 mg to about 200 mg per day. A preferred DPP4 inhibitor is sitagliptin, and it will be employed at doses from about 50 mg to about 300 mg per day. Slow release metformin hydrochloride will be administered at doses of about 300 mg to about 2000 mg per day. It is available commercially in tablets which contain 500 mg, 750 mg and 1000 mg of active agent. The number of the dosages given are administered depends of the nature of the disease and the conditions of the patients but can be given up to two times a day or more.

The invention provides compositions of anti-diabetic combinations, for example, DPP4 inhibitor and a slow release biguanide, and a method of treating diabetes and controlling glycemic conditions comprising administering to a patient in need of treatment an effective amount of a DPP4 inhibitor and slow release biguanide. When the DPP4 inhibitor and a slow release biguanide are formulated together, the compositions will contain from about 1 and to about 1000 by weight DPP4 inhibitor and about 100-2000 mg of biguanide. For example, a typical two-way composition includes 50 mg of sitagliptin and 500 mg of metformin. The compositions may contain common excipients and carriers such as starch, sucrose, polymers, talc, gelatin, methylcellulose, and magnesium stearate. The compositions will normally be made for oral administration, for instance as tablets or capsules, but also may be in the form of aqueous suspensions or solutions, suppositories, slow release forms, for example employing an osmotic pump, skin patch, or the like.

### PHARMACEUTICAL COMPOSITION

The present invention further concerns a pharmaceutical composition or dosage form comprising a slow release biguanide as the first active ingredient and a DPP4 inhibitor as the second active ingredient. Further, biguanide is preferably a metformin or its pharmaceutically acceptable salt thereof and is delivered in a controlled release manner from a tablet core, preferably an osmotic tablet core with or without a gelling or swelling polymer. The tablet core should include the biguanide and at least one pharmaceutically acceptable excipient. In one embodiment of the present invention the tablet core includes the biguanide, a binding agent and an absorption enhancer, and the tablet core is preferably coated with a polymeric coating to form a membrane around the tablet and drilled to create one passageway on each side of the membrane. The second active ingredient comprises a DPP4 inhibitor or its pharmaceutically equivalent salt, and is preferably applied to the membrane of the tablet core and provides for either immediate or controlled release of said DPP4 inhibitor.

In a preferred embodiment, the use of an absorption enhancer is optional and it can be any type of absorption enhancer commonly known in the art such as a fatty acid, a surfactant (anionic, cationic, amphoteric), a chelating agent, a bile salt or mixtures thereof. Examples of some preferred absorption enhancers are lecithin, fatty acids such as capric acid, oleic acid and their monoglycerides, surfactants such as sodium lauryl sulfate, sodium taurocholate and polysorbate 80, chelating agents such as citric acid, phytic acid, ethylenediamine tetra acetic acid (EDTA) and ethylene glycol-bis(.beta.-amino ethyl ether)-N,N,N,N-tetra acetic acid (EGTA). The core may comprise approximately 0 to about 20% of the absorption enhancer based on the total weight of the core and most preferably about 2% to about 10% of the total weight of the core.

In one embodiment of the present invention, the core of the present invention is preferably formed by granulating a biguanide with a binding agent and compressing the granules with the addition of a lubricant and absorption enhancer into a tablet and this embodiment doesn't use a gelling or swelling polymer. The core may also be formed either by dry granulating the core ingredients by passing them through a roller compactor and compressing the granules with the addition of a lubricant into tablets or by direct compression. It can also be achieved using other commonly known granulation procedures that are known in the art. This is only an example as, other excipients such as lubricants, pigments or dyes may also be employed in the formulation of the subject invention.

A membrane or sustained release coating is used as a coat in the core as outlined in this specification. Materials that are useful in forming the membrane or slow release coating are ethylcellulose, cellulose esters, cellulose diesters, cellulose triesters, cellulose ethers, cellulose ester-ether, cellulose acylate, cellulose diacylate, cellulose triacylate, cellulose acetate, cellulose diacetate, cellulose triacetate, cellulose acetate propionate and cellulose acetate butyrate. Other suitable polymers are described in U.S. Pat. Nos. 3,845,770; 3,916,899; 4,008,719; 4,036,228 and 4,612,008 which are incorporated herein by reference. Cellulose acetate, comprising an acetyl content of 39.3 to 40.3%, and is commercially available from Eastman Fine Chemicals, is the most preferred membrane or slow release coating

Further in an alternative embodiment, a flux-enhancing agent can also be included in the membrane or slow release coating can include one of the above-described polymers. The flux enhancing agent can increase the volume of fluid imbibed into the core to enable the dosage form to dispense substantially all of the biguanide through the passage and/or the porous membrane.

The flux-enhancing agent can be a water-soluble material or an enteric material. Examples of the preferred materials that are useful as flux enhancers include but not limited to sodium chloride, potassium chloride, sucrose, sorbitol, mannitol, polyethylene glycols (PEG), propylene glycol, hydroxypropyl cellulose, hydroxypropyl methycellulose, hydroxypropyl methycellulose phthalate, cellulose acetate phthalate, polyvinyl alcohols, methacrylic acid copolymers, poloxamers (such as LUTROL F68, LUTROL F1 27, LUTROL F108 which are commercially available from BASF) and mixtures thereof. A preferred flux-enhancer used in this invention is PEG 400.

The flux enhancer may also be a water soluble drug such as metformin or its pharmaceutically acceptable salts, or the flux enhancer may be a drug that is soluble under intestinal conditions. If the flux enhancer is a drug, the present pharmaceutical composition has an added advantage of providing an immediate release of the drug that has been selected as the flux enhancer. The flux enhancing agent dissolves or leaches from the membrane or sustained release coating to form channels in the membrane or sustained release coating which enables fluid to enter the core and dissolve the active ingredient. In the preferred embodiment, the flux enhancing agent comprises approximately 0 to about 40% of the total weight of the coating, most preferably about 2% to about 20% of the total weight of the coating.

A commonly known excipient such as a plasticizer may also be used for preparing the membrane or slow release coating Some commonly known plasticizers include but not limited to adipate, azelate, enzoate, citrate, stearate, isoebucate, sebacate, triethyl citrate, tri-n-butyl citrate, acetyl tri-n-butyl citrate, citric acid esters, and all those described in the Encyclopedia of Polymer Science and Technology, Vol. 10 (1969), published by John Wiley & Sons. The preferred plasticizers are triacetin, acetylated monoglyceride, grape seed oil, olive oil, sesame oil, acetyltributylcitrate, acetyltriethylcitrate, glycerin sorbitol, diethyloxalate, diethylmalate, diethylfumarate, dibutylsuccinate, diethylmalonate, dioctylphthalate, dibutylsebacate, triethylcitrate, tributylcitrate, glyceroltributyrate and the like. Though the exact amount used depends on the type of plasticizer used, typically amounts from about 0 to about 25% are used, and preferably about 2% to about 15% of the plasticizer can be used based upon the total weight of the membrane or sustained release coating.

Generally, the membrane or slow release coating around the core will comprise from about 1% to about 10% and preferably about 2% to about 5% based upon the total weight of the core and coating.

The membrane or sustained release coating surrounding the core further comprises a passage that will allow for controlled release of the drug from the core in a preferred embodiment. As used herein the term passage includes an aperture, orifice, bore, hole, weakened area or a credible element such as a gelatin plug that erodes to form an osmotic passage for the release of the biguanide from the dosage form. Passage used in accordance with the subject invention are well known and are described in U.S. Pat. Nos. 3,845,770; 3,916,899; 4,034,758; 4,077,407; 4,783,337 and 5,071,607.

The present invention provides a combination that includes a DPP4 inhibitor that is independent of the biguanide. This constitutes the second active ingredient and may be formulated to provide an immediate release of the DPP4 inhibitor. In one embodiment of the present invention the DPP4 inhibitor is applied in the form of a layer to a controlled or slow released core comprising the a biguanide as a layer using a binder and other conventional pharmaceutical excipients such as absorption enhancers, surfactants, plasticizers, antifoaming agents and combinations of the foregoing. An absorption enhancer may be present in the DPP4 inhibitor layer in an amount up to about 30% w/w in comparison to the weight of the DPP4 inhibitor. A binding agent may be present in an amount up to 150% w/w of the DPP4 inhibitor. A second active ingredient immediate release formulation may be incorporated into a single dosage form by coating onto the membrane or slow release coating of the dosage form by conventional methods. Alternatively, it may also be incorporated by any pharmaceutically acceptable method into a single dosage form with the first active ingredient. The incorporation of the second active ingredient may be performed, among others, by commonly used processes selected from the group consisting of drug layering, lamination, dry compression, deposition and printing.

When the DPP4 inhibitor is coated onto a membrane or slow release coating of an osmotic tablet core, the DPP4 inhibitor coating should be applied from a coating solution or suspension that employs an aqueous solvent, an organic solvent or a mixture of an aqueous and an organic solvent. Typical organic solvents include acetone, isopropyl alcohol, methanol and ethanol. Whenever a mixture of aqueous and organic solvents is employed, the ratio of water to organic solvent should be in the range from 98:2 to 2:98, preferably 50:50 to 2:98, mort preferably 30:70 to 20:80 and most preferably from about 25:75 to about 20:80. When a mixed solvent system is employed, the amount of binder required for coating the DPP4 inhibitor onto the membrane or a slow release coating may be reduced. For example, successful coatings have been obtained from a mixed solvent system where the ratio of binder to DPP4 inhibitor is 1:9 to 1:11. Although acceptable coatings can be obtained when the DPP4 inhibitor coat is applied directly to the membrane or slow release coating, a preferred approach is to first coat the membrane or slow release coating with a seal coat prior to the application of the DPP4 inhibitor coating. The DPP4 inhibitor coating solution or suspension may also contain a surfactant and a pore forming agent such as sodium chloride, potassium chloride, sucrose, sorbitol, mannitol, polyethylene glycols (PEG), propylene glycol, hydroxypropyl cellulose, hydroxypropyl methycellulose, hydroxypropyl methycellulose phthalate, cellulose acetate phthalate, polyvinyl alcohols, methacrylic acid copolymers, poloxamers. In an alternative embodiment, the pharmaceutical composition of the present invention may also comprise an effective immediate release amount of the biguanide. The effective immediate release amount of biguanide may be coated onto the membrane or slow release coating of the dosage form or it may be incorporated into the membrane or slow release coating.

In addition, various diluents, excipients, lubricants, dyes, pigments, dispersants, etc., which are disclosed in Remington's Pharmaceutical Sciences (1995), may be used to optimize the above listed formulations of the subject invention.

Biguanides, such as metformin are commonly administered in dosage forms containing 500 mg, 750 mg, 850 mg, and 1000 mg. DPP4 inhibitors, for example sitagliptin, is commonly administered in dosage forms containing 25 mg, 50 mg and 100 mg⁶. The present invention is intended to encompass the above listed therapeutic combinations, without providing a specific example of each possible combination of compounds and their respective dosage amounts.

A preferred embodiment of the pharmaceutical composition form, using Sitagliptin Phosphate as described in U.S. Pat. No. 6,303,661 will have the following composition (Table 1):

TABLE 1

| | Range percent | Preferred Range % |
|---|---|---|
| First Active | | |
| Ingredient | | |
| Drug | 50-98% | 75-95% |
| Binder | 0.1-40% | 3-15% |
| Absorption Enhancer | 0-20% | 2-10% |
| Lubricant | 0-5% | 0.5-1% |
| Coat | | |
| Polymer | 50-99% | 75-95% |
| Flux Enhancer | 0-40% | 2-20% |
| Plasticizer | 0-25% | 2-15% |
| Second Active | | |
| Ingredient | | |
| Drug | 0.1-20% | 1-10% |
| Binder | 0.1-20% | 1-15% |
| Surfactant | 0-20% | 0.1-15% |
| Pore Former | 0-25% | 0.1-15% |
| Polymer (Optional) | 0-30% | 0.1-20% |

### EXAMPLE 1

The Table 2 shows the representative example of a pharmaceutical composition of a slow release comprising biguanide and a DPP inhibitor. We have used the pharmaceutical composition comprising 500 mg metformin hydrochloride and 50 mg of Sitagliptin Phosphate as an example. However it is possible for a person skilled in the art to make variations of this composition by modifying the drugs, excipients, the quantities and the process of manufacturing them.

TABLE 2

| Amount | |
|---|---|
| mg/tablet | |
| First Active Ingredient | |
| Metformin HCl | 500.0 |
| Povidone K 301 USP | 30.0 |
| Sodium Lauryl Sulfate | 26.0 |
| Magnesium Stearate | 2.8 |
| Seal Coat | |
| Opadry Clear (YS 1-7006) | 24.0 |
| Semi permeable coat | |
| Cellulose Acetate (398-10) NF | 7.6 |
| Triacetin | 0.5 |
| PEG 400 | 0.9 |
| Seal coat | |
| opadry Clear (YS 1-7006) | 5.0 |
| Second Active Ingredient | |
| Sitagliptin Phosphate | 50.0 |
| Povidone K 30 USP | 1.5 |
| Lactose Monohydrate | 35.0 |
| Sodium starch Glycolate | 12.5 |
| Poloxamer 188 | 6.0 |
| HPMC | 2.5 |
| PEG 8000 | 0.4 |
| Titanium Dioxide | 0.4 |
| Wax | 0.2 |

The dosage forms prepared according to the present invention exhibit the dissolution profile shown Table 3 when tested in a USP Type 2 apparatus at 75 rpm in 900 ml of simulated intestinal fluid (pH 7.5 phosphate buffer) and at 37° C.

TABLE 3
Dissolution Profile

| | | |
|---|---|---|
| Time hours | Percent Release | Preferred Range |

Biguanide

| | | |
|---|---|---|
| 2 | 0-25% | 0-15% |
| 4 | 10-45% | 20-40% |
| 8 | 30-90% | 45-90% |
| 12 | >50% | >60% |
| 16 | >60% | >60% |
| 20 | >70% | >70% |

DPP4 Inhibitor

| | | |
|---|---|---|
| 1 | >85% | >85% |

It has been discovered that the selection of the excipients for use in the DPP4 ingredient layer of the dosage form can greatly affect the release characteristics, potency and stability of the DPP4 inhibitor. Therefore, in an alternate embodiment of the present invention, the composition of the DPP4 inhibitor component of the present invention should be selected so that not less than 85%, preferably not less than 90% and most preferably not less than 95% of the DPP4 inhibitor is released from the dosage form within 120 minutes, preferably within 90 minutes and most preferably within 60 minutes when tested according to the United States Pharmacopeia (USP) 26, with Apparatus 1 at 100 rpm, 37° C. and 900 ml of 0.3 M KCl-HCl Buffer, pH 2.0.

Further the excipients for use in the DPP4 inhibitor layer of the dosage form should be selected so that the total DPP4 inhibitor related compounds or impurities in the final dosage form are not more than 0.6%, preferably not more than 0.5% and most preferably not more than 0.25% and each individual DPP4 inhibitor related compound or impurity in the final dosage form is not more than 0.25%, preferably not more than 0.2% and most preferably not more than 0.1%. The DPP inhibitor related compounds or impurities in the final dosage form are determined by High Performance Liquid Chromatography (HPLC) using a YMC-ODS-AQ, 5 .mu.m, 120 .ANG., 4.6.times.250 mm or equivalent column, a 0.1 M ammonium acetate buffer:acetonitrile:glacial acetic acid (25:25:1) mobile phase, about a 40 .mu.L injection volume, 0.7 mL/min flow rate, 25° C. column temperature and 269 nm wavelength for the UV detector.

Manufacturing of Metformin Hydrochloride Core

By way of an Example of the invention claimed in this specification, the slow-release tablet containing 500 mg of metformin HCl and 50 mg sitagliptin phosphate is prepared using a three step process: 1) Granulation, 2) Tableting and 3) Membrane coating process. An optional Seal Coating may be done on the core tablet. These specific steps are described below:

Granulation: Metformin hydrochloride was screened using a size reduction and screening equipment (Comil screener) and was further fluidized using a commercially available powder coater granulator (Glatt 60). It was sprayed with Povidone solution that was prepared in a steel tank using water as solvent. The spraying was carried at about 2.5 bar pressure by varying the pump rate from 0-15 minutes for a target of 500 g/minute to achieve a target of about 1200 g/.minute in the final phase. Granules are dried until an LOD of less than 2% and passed through a screener (Comil 1143/75).

Tableting: Metformin hydrochloride was mixed with sodium lauryl sulfate in a blender (Slant-Cone: 30 minutes). Magnesium stearate was screened and blended with metformin hydrochloride-sodium lauryl sulfate mixture. The homogenized mixture was compressed into tablets using standard procedure. The metformin hydrochloride core tablets weighted from 650 mg to 800 mg with a frigidity of less than 1%.

Seal coating: Seal coating of the above prepared metformin core tablets was done by spraying (O'Hara Lab Coat Pan Coater) a solution of either Opadry or other water soluble substitute coating material. The spraying was don at a temperature of 46-47° C., atomization pressure of 40-60 psi at a spray rate of 180 grams per minute/three guns. The pan speed was at 4-8 rpm and air volume of 1000+/−100. The seal coated metformin hydrochloride had a theoretical coating of 2.5-5.0%

Membrane coating: Cellulose acetate was mixed with acetone to prepare a clear solution. Polyethylene glycol 400 was added this mixture and the resulting solution was further mixed with triacetin. The seal coated metformin hydrochloride tablets were fluidized using a Glatt coater. The cellulose acetate solution was sprayed onto the fluidized seal coated metformin hydrochloride tablets at an atomization pressure of 2.5 bars, using an air volume of 1700 CFM, at spraying rate of about 450 g/ml to achieve coating target of 1.3%. Membrane coated tablets were dried sequentially at temperature of 21° C. and 40° C. An orifice was made on the membrane coated tablets using laser with an average diameter of 0.4 to 0.5 mm with micrometer ranging from 6 to 7 mm. Laser was operated with pulse width of 165+/−65 and a pulse delay of around 340+/−100 respectively.

Manufacturing Process of Sitagliptin Phosphate Coating

The above prepared membrane coated metformin hydrochloride tablets were further seal coated with Opadry Clear (YS-1-7006) solution using standard coater like O'Hara pan coater tip set at 4″ at a spray rate of 25 mL/gun/min, exhaust temperature of around 45° C., an atomization pressure from 10-35 psi at a pan speed of 5-8 rpm, using airflow 350 CFM. The sitagliptin coating solution was prepared carefully and slowly by dissolving Lutrol F-68 in water. Similarly the povidone K-30 in water solution was prepared separately and was mixed with spray dried lactose monohydrate. Following the addition of lactose, sitagliptin was first dispersed in the above prepared Lutrol solution with constant stirring and finally sodium starch glycolate was added into the coating solution. The sitagliptin coating was applied to seal coated 500 mg metformin hydrochloride membrane coated tablets using the above mentioned coater at identical conditions. Over this 50 mg sitagliptin coated seal coated 500 mg metformin hydrochloride membrane coated tablets, color coating was done using similar coater and identical conditions mentioned above.

Finally, color coated tablets were dried and polished using Cindrella wax and the finished final tablets were packaged in a HDPE bottle with a suitable desiccant and subjected appropriate stability and clinical studies.

### EXAMPLE 2

The Table 4 shows the representative example of a pharmaceutical composition of a slow release comprising biguanide and a DPP inhibitor. We have used the pharmaceutical composition comprising 1000 mg metformin hydrochloride and 100 mg of Sitagliptin Phosphate as an example.

TABLE 4

| | Amount |
|---|---|
| | mg/tablet |
| First Active Ingredient | |
| Metformin HCl | 1000.0 |
| Povidone K 301 USP | 78.0 |
| Sodium Lauryl Sulfate | 51.7 |
| Magnesium Stearate | 5.7 |
| Seal Coat | |
| Opadry Clear (YS 1-7006) | 47.0 |
| Semi permeable coat | |
| Cellulose Acetate (398-10) NF | 15.5 |
| Triacetin | 0.9 |
| PEG 400 | 1.8 |
| Seal coat | |
| Opadry Clear (YS 1-7006) | 9.0 |
| Second Active Ingredient | |
| Sitagliptin Phosphate | 100.0 |
| Povidone K 30 USP | 3.0 |
| Lactose Monohydrate | 70.0 |
| Sodium starch Glycolate | 25.0 |
| Poloxamer 188 | 12.0 |
| HPMC | 5.0 |
| PEG 8000 | 0.8 |
| Titanium Dioxide | 0.8 |
| Wax | 0.4 |

The pharmaceutical composition comprising sitagliptin phosphate 100 mg and slow release metformin hydrochloride 1000 mg was manufactured as in Example 1.

The following combinations were tested in vivo each in a cross over study with the combination of Glucophage 1000 mg (commercially available metformin XR 100 mg) and Januvia 100 mg (commercially available sitagliptin phosphate 100 mg). The in vivo test employed 14 healthy volunteers and each dosed after evening meal.

The Pharmacokinetic parameters of metformin hydrochloride and sitagliptin phosphate are listed in Table 5 and Table 6 respectively

TABLE 5

| | Metformin Parameter | | | | |
|---|---|---|---|---|---|
| | | | | AUC | |
| | Mean AUCO- | | | Ratio | Cmax |
| | 12 | Mean Cmax | Mean Tmax | (Test/ | (Test/ |
| Combination drug/day | (ng.hr/ML) | (ng/ML) | hr | BID) | BID) |
| 2 Glucophage 500 mg + 2 Januvia 50 mg (BID) | 10246 | 1454 | 3 | 1 | 1 |
| Fortamet 1000 mg + Januvia 100 mg | 11900 | 1424 | 6.3 | 1.16 | 0.98 |
| Glumetza 1000 mg + Januvia 100 mg | 12580 | 1293 | 9 | 1.23 | 0.89 |
| Glucophage XR 1000 mg + Januvia 100 mg | 14793 | 1648 | 7 | 1.44 | 1.13 |
| Example 2 | 12345 | 1353 | 6.4 | 1.20 | 0.93 |

TABLE 6

| | Sitagliptin Parameter | | | | |
|---|---|---|---|---|---|
| | | | | AUC | |
| | Mean AUCO- | | Mean | Ratio | Cmax |
| | 12 | Mean Cmax | Tmax | (Test/ | (Test/ |
| Combination drug/day | (ng.hr/ML) | (ng/ML) | hr | BID) | BID) |
| 2 Glucophage 500 mg + 2 Januvia 50 mg (BID) | 8.43 | 938 | 2.6 | 1 | 1 |
| Fortamet 1000 mg + Januvia 100 mg | 7.9 | 910 | 3 | 0.94 | 0.97 |
| Glumetza 1000 mg + Januvia 100 mg | 8.9 | 980 | 1.9 | 1.06 | 1.04 |
| Glucophage XR 1000 mg + Januvia 100 mg | 9.05 | 895 | 2.25 | 1.07 | 0.95 |
| Example 2 | 8.3 | 940 | 2.4 | 0.98 | 1.00 |

### METHOD OF ADMINISTRATION

The present inventions disclosed in this specification further include a method of treating diabetes and diabetes related disorders. This was established using a well controlled human clinical trial. The method of treating diabetes employing a combination provided by this invention has been established in a long-term controlled clinical evaluation. A typical study determined the efficacy of DPP inhibitor, biguanide alone and a combination of a DPP4 inhibitor and a slow release biguanide; for example metformin for the treatment of non-insulin dependent diabetes mellitus (NIDDM). This trial targeted the segment of the type 2 diabetes population wherein the disease state has progressed to a point where maximum doses of metformin. These patients are at a stage where the maximally stimulated pancreatic insulin secretion does not keep up with the increasing demand. Since the un-stimulated (metformin) insulin secretory capacity of the beta cells is very low in this population, reversing insulin resistance alone would be of partial benefit. Therefore, maintaining a level of stimulated insulin secretion with a metformin while adding sitagliptin to improve insulin sensitivity could provide a level of glycemic control unattainable by either medication alone.

A primary objective of the study was to assess the efficacy of a DPP inhibitor in combination with a slow release biguanide in patients with type 2 diabetes by comparing changes in markers of glycemic and lipid homeostasis over six months of treatment.

The effect of treatment on the pattern of post-prandial glucose tolerance (standard 2-hour meal tolerance test) was determined in a subset of patients. A brief summary of the results of the 24 week-month, 5-center clinical trial study in 100 patients is presented below.

### CLINICAL TRIALS

1. Drugs:
Sitagliptin: Januvia 50 mg,
Immediate release Metformin Hydrochloride: Glucophage 500 mg,
Slow Release Metformin: a) Example 1, b) Glumetza XL 500 mg, c) Fortamet 500 mg and d) Glucophage XR 500 mg

2. Treatment Combination

| Treatment | Drugs per day per patient |
|---|---|
| 1. Treatment A; | Januvia 100 mg |
| 2. Treatment B: | Glucophage 1000 mg |
| 3. Treatment C; | Januvia 100 mg + Glucophage XR 1000 mg |
| 4. Treatment D: | Januvia 100 mg + Fortamet 1000 mg |
| 5. Treatment E: | Januvia 100 mg + Glumetza 1000 mg |
| 6. Treatment: F | Januvia 100 mg + Example 1 Fixed dose |

3. Dosage:

The administered dosage comprised either sitagliptin (50 mg) or an immediate release metformin hydrochloride 500 mg or a combination of sitagliptin phosphate (50 mg) and slow release metformin (500 mg) selected from Treatments C, D, E and F was administered twice a day to the patients in a long-term clinical trial.

4. Clinical Parameters:

The objectives of the invention were set by measuring following two parameters in the clinical trials
1. Fasting Plasma Glucose: Changes in fasting plasma glucose (FPG) during sitagliptin monotherapy and during the combination comprising sitagliptin and a slow release metformin hydrochloride. The fasting plasma glucose test is a carbohydrate metabolism test which measures plasma, or blood, glucose levels after a fast. Fasting stimulates the release of the hormone glucagon, which in turn raises plasma glucose levels. In people without diabetes, the body will produce and process insulin to counteract the rise in glucose levels. In people with diabetes this does not happen, and the tested glucose levels will remain high.
2. Hemoglobin: Changes in hemoglobin Alc (HbA_{1c}) during 3 months of monotherapy of sitagliptin and after an additional 3 months of combination therapy (sitagliptin phosphate and slow release metformin hydrochloride). The hemoglobin A1c test shows if a person's blood sugar is close to normal or too high.
5. General Methods:
   a. Change measurement:
   The trial used the methodology to compare the baseline clinical laboratory parameters with the values at the end of the study or last visit to identify any abnormal trends. The percent of patients.with increases or decreases in laboratory values were calculated based on the number of patients at risk for changes outside of the reference range. Here the patients with low or high values at baseline were not considered at risk for a decrease or increase, respectively. No clinically adverse trends were noted in any laboratory parameter. However, dramatic decrease in urine glucose for all combination therapy groups was evident indicating significant improvement. Laboratory results were then reviewed for these particular patients to determine which patients actually had clinically important changes in a given laboratory parameter. Minimal changes occurred within any laboratory parameter across all treatments.
   A greater number of patients treated with sitagliptin and slow release metformin combination therapy than with either sitagliptin or metformin monotherapy had laboratory changes meeting clinically meaningful change criteria.
   b. Adverse Events:
Among patients treated with sitagliptin and a slow release metformin combination therapy, about 10% of patients had adverse events compared with 8% and 10% of patients treated with sitagliptin and metformin monotherapy. Patients treated with combination therapy with different treatments E, D, E and F did not have statistically significant variation.
6. Laboratory Parameters
Hematology: Minimal changes occurred with any of the hematological parameters. Changes that met criteria for possible clinical importance were increases or decreases within the normal range or transient changes that subsequently resolved.
Patients meeting clinically meaningful changes in hematology parameters are classified based on the reasons.
Total Hemoglobin/Hematocrit Changes: 4%
   a. Transient decreases which returned to baseline levels: 1.0%
   b. Below normal limits throughout the trial: 1.5%
   c. Miscellaneous reasons other than trial: 3.1%
The analysis of the patient laboratory data, it was determined no patient experienced clinically important decreases in any hematological parameter that can be directly attributable to sitagliptin. Among the liver enzymes analysis, it was found only 2.3 had any clinically meaningful elevations in the ALT and AST. Further analysis again concluded that sitagliptin was not responsible for the variations
7. Results:

The objectives of the inventions are met by the following results from the clinical trials

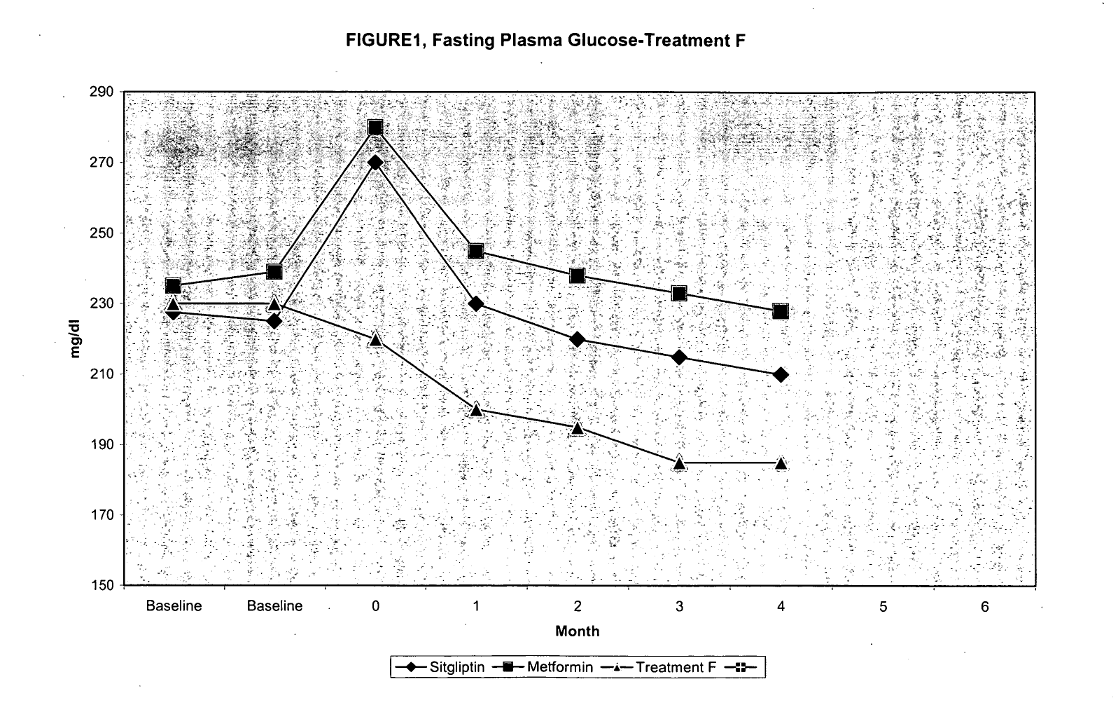

FIG. 1: Change in fasting plasma glucose (FPG) (+/−) SEM) during sitagliptin, metformin hydrochloride monotherapy and an additional three months of Treatment F comprising sitagliptin phosphate and slow release metformin hydrochloride fixed dose combination.

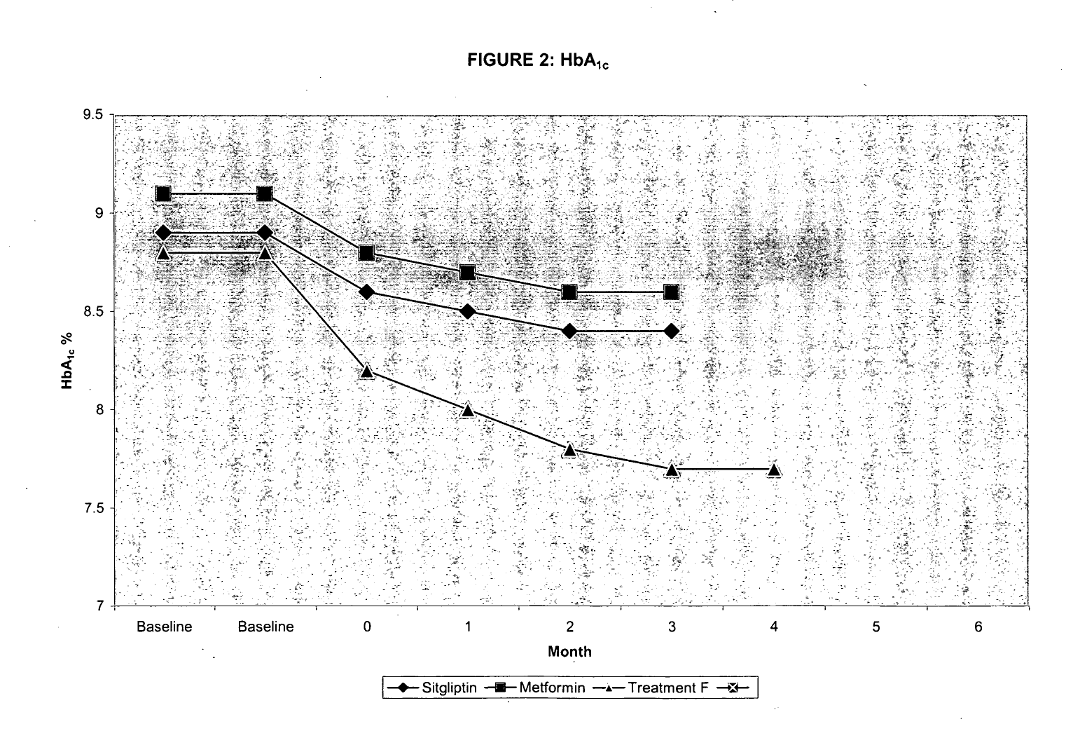

FIG. 2: Change in hemoglobin A1c (HbA_{1c}) (+/−.SEM) during 3 months of sitagliptin and metformin hydrochloride monotherapy, and after an additional 3 months of Treatment F comprising sitagliptin phosphate and slow release metformin hydrochloride fixed dose combination.

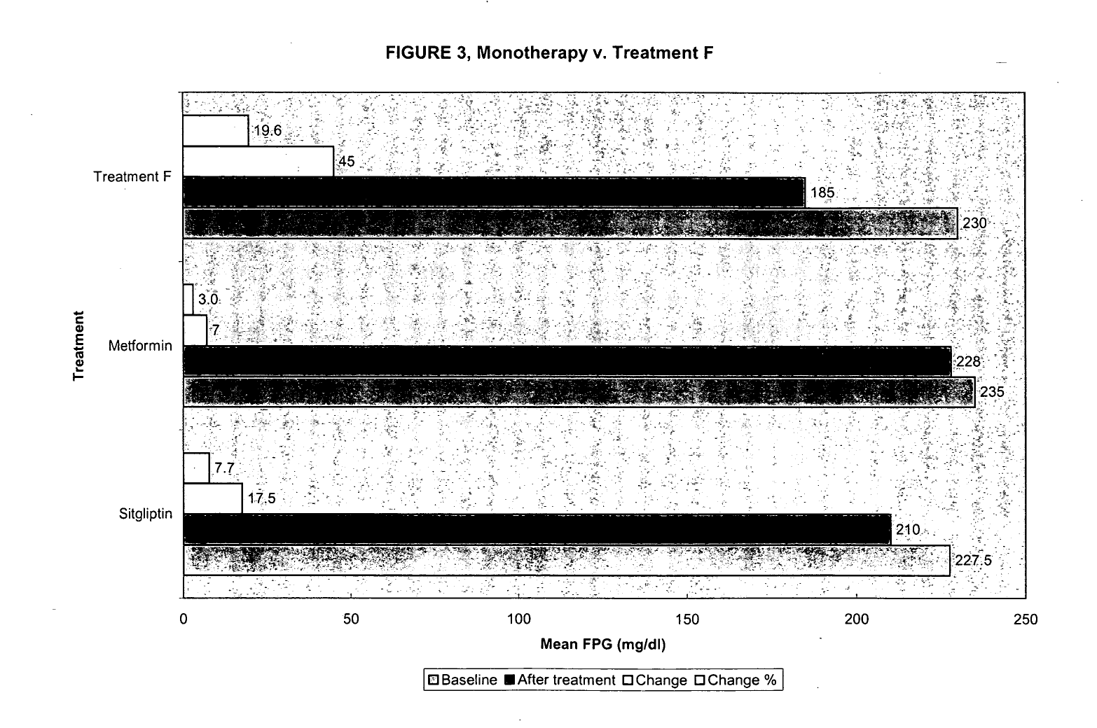

FIG. 3: Change in mean FPG for sitagliptin, metformin monotherapy and after three months additional combination therapy using Treatment F comprising sitagliptin phosphate and slow release metformin hydrochloride fixed dose combination.

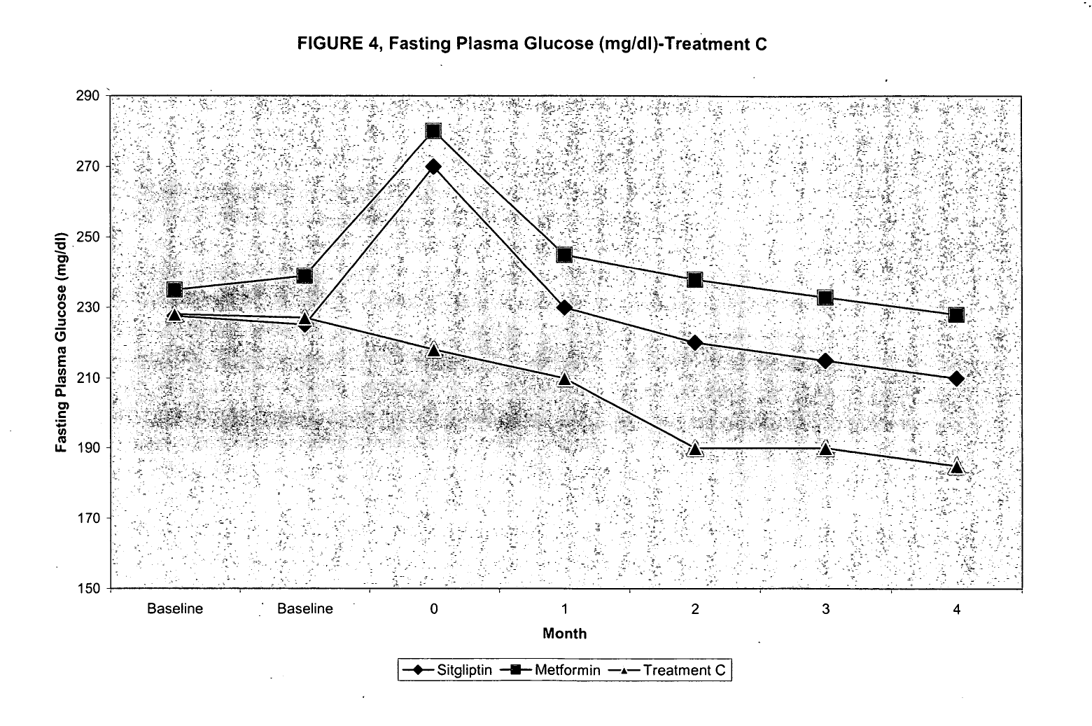

FIG. 4: Change in fasting plasma glucose (FPG) (+/−) SEM) during sitagliptin, metformin hydrochloride monotherapy and an additional three months of Treatment C comprising the co-administration of sitagliptin phosphate and Glucophage XR.

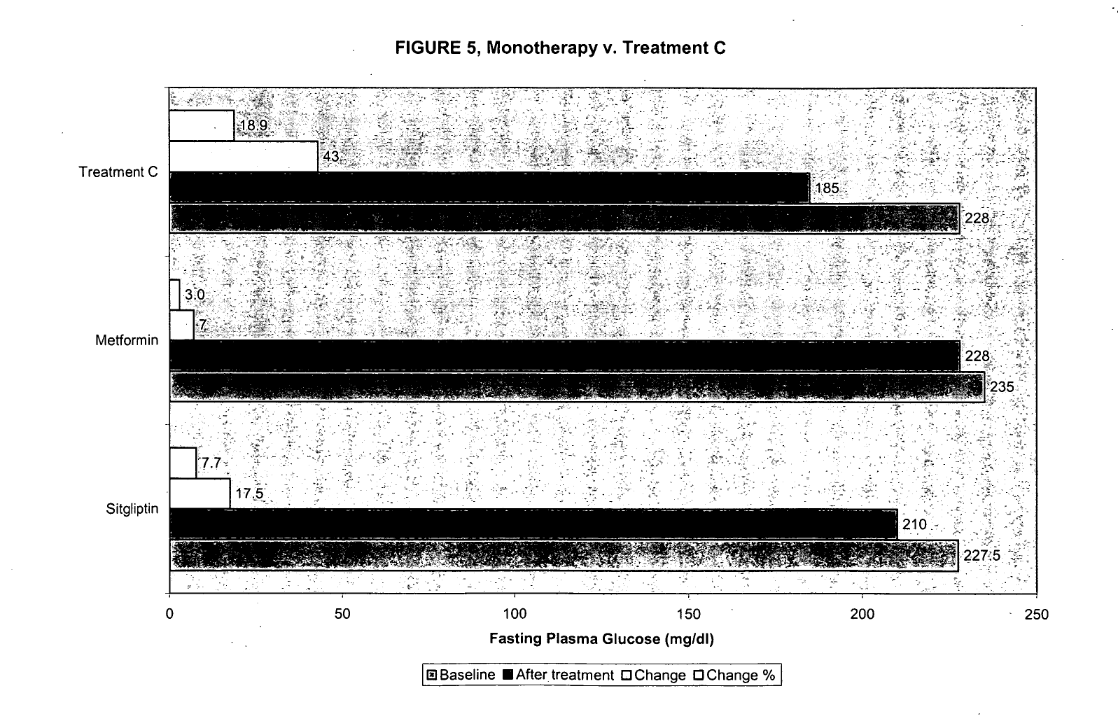

FIG. 5: Change in mean FPG for sitagliptin, metformin monotherapy and after three months additional combination therapy using Treatment C comprising the co-administration of sitagliptin phosphate and Glucophage XR.

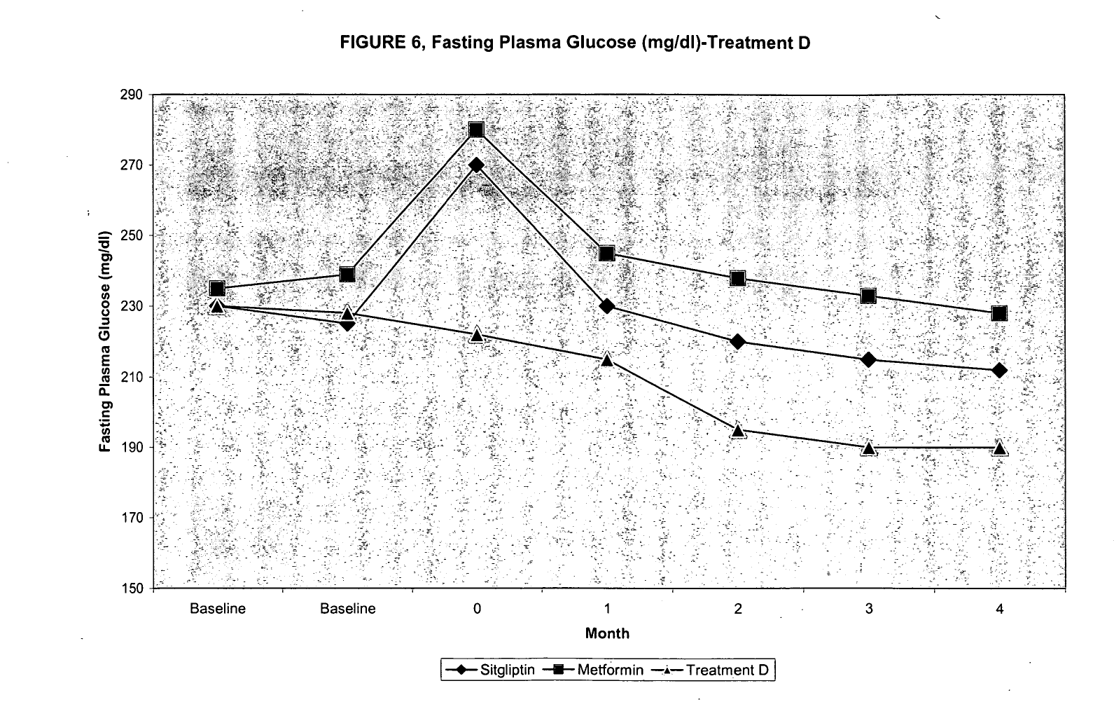

FIG. 6: Change in fasting plasma glucose (FPG) (+/−) SEM) during sitagliptin, metformin hydrochloride monotherapy and an additional three months of Treatment D comprising the co-administration of sitagliptin phosphate and Fortamet

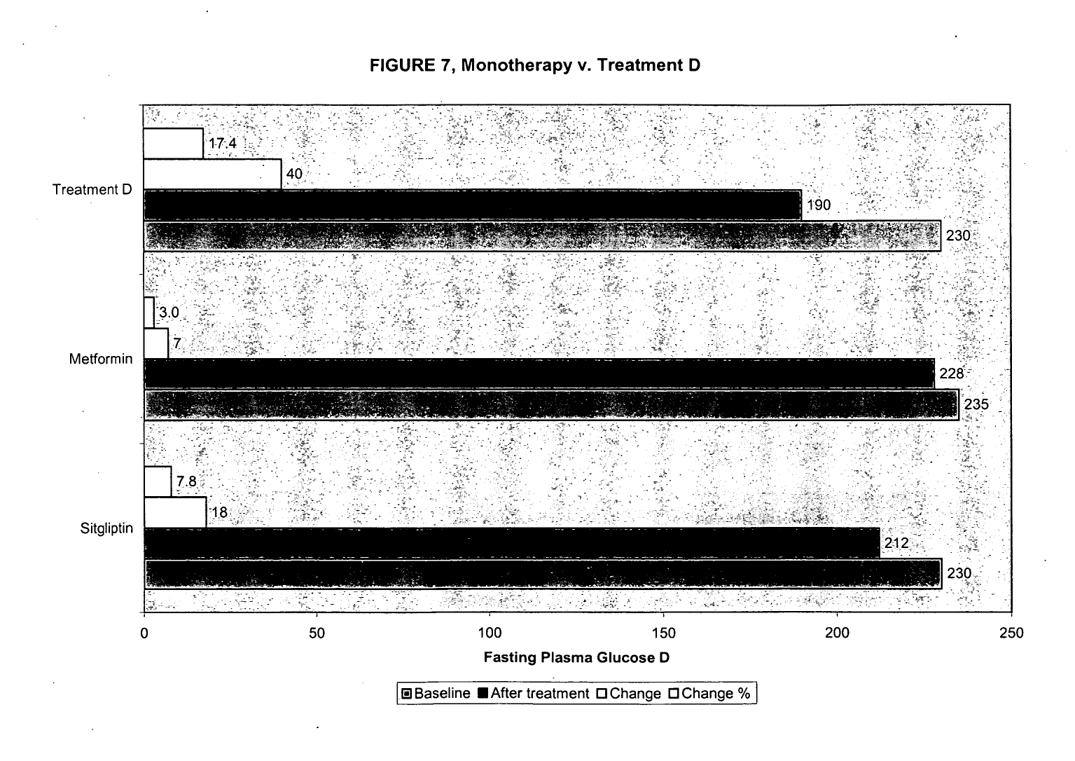

FIG. 7: Change in mean FPG for sitagliptin, metformin monotherapy and after three months additional combination therapy using Treatment D comprising the co-administration of sitagliptin phosphate and Fortamet

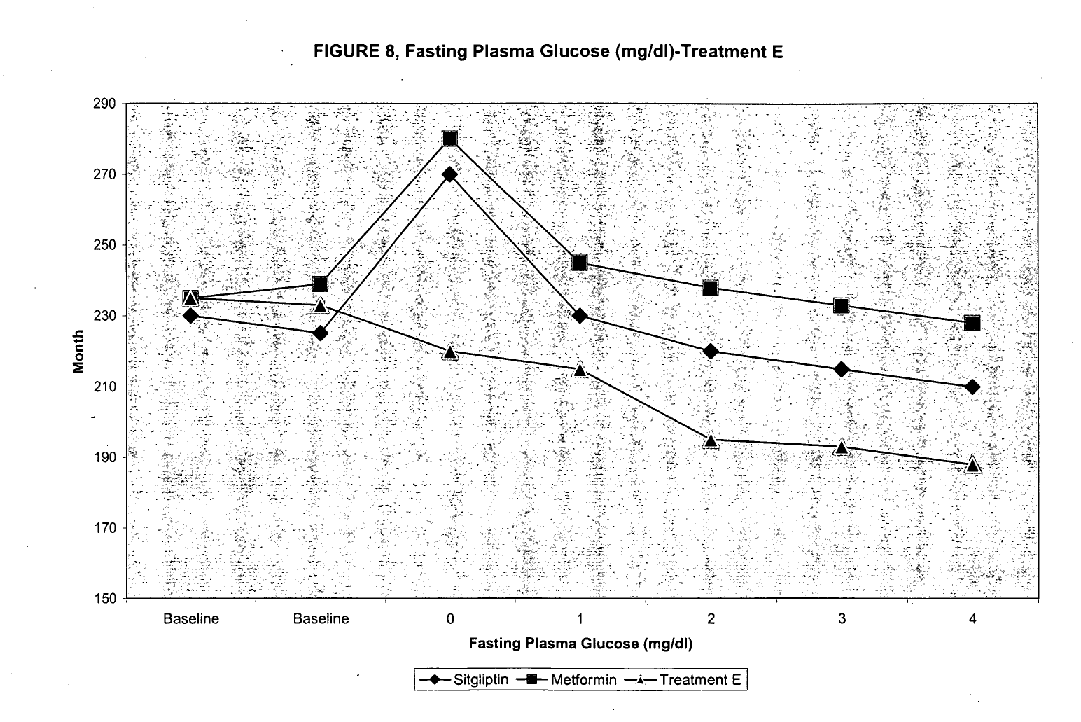

FIG. 8: Change in fasting plasma glucose (FPG) (+/−) SEM) during sitagliptin, metformin hydrochloride monotherapy and an additional three months of Treatment E comprising the co-administration of sitagliptin phosphate and Glumetza

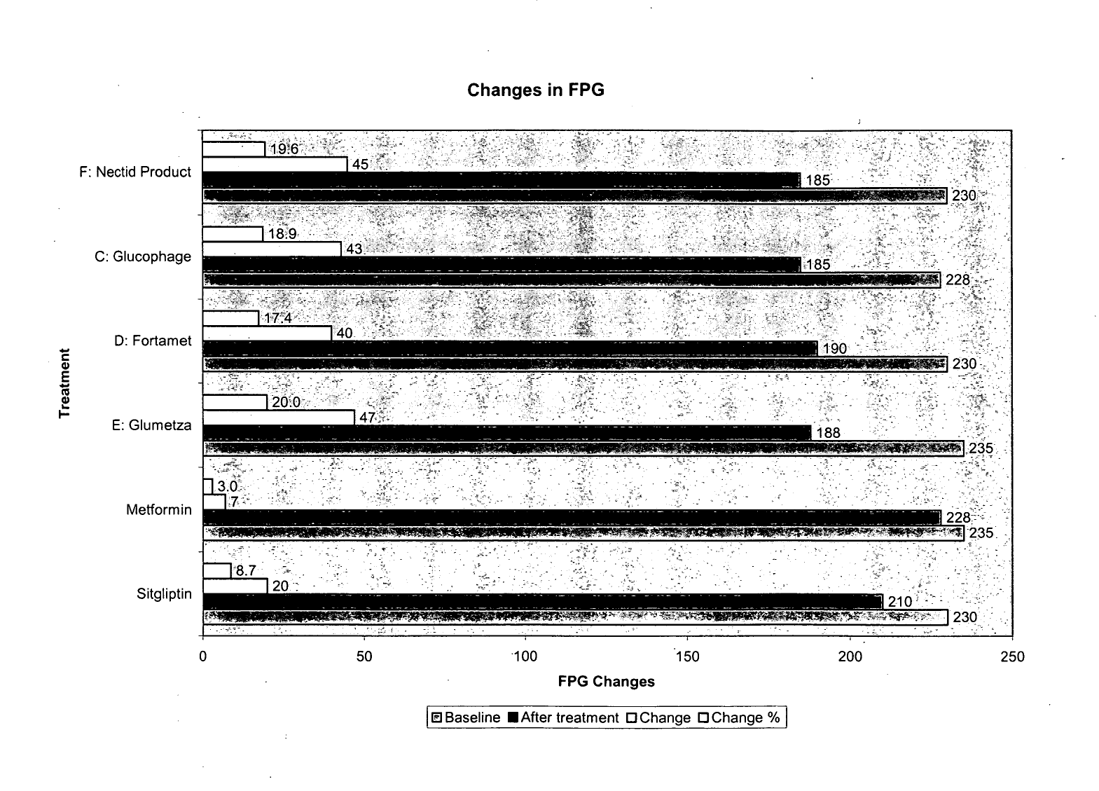

FIG. 9: Change in mean FPG for sitagliptin, metformin monotherapy and after three months additional combination therapy using Treatment E comprising the co-administration of sitagliptin phosphate and Glumetza

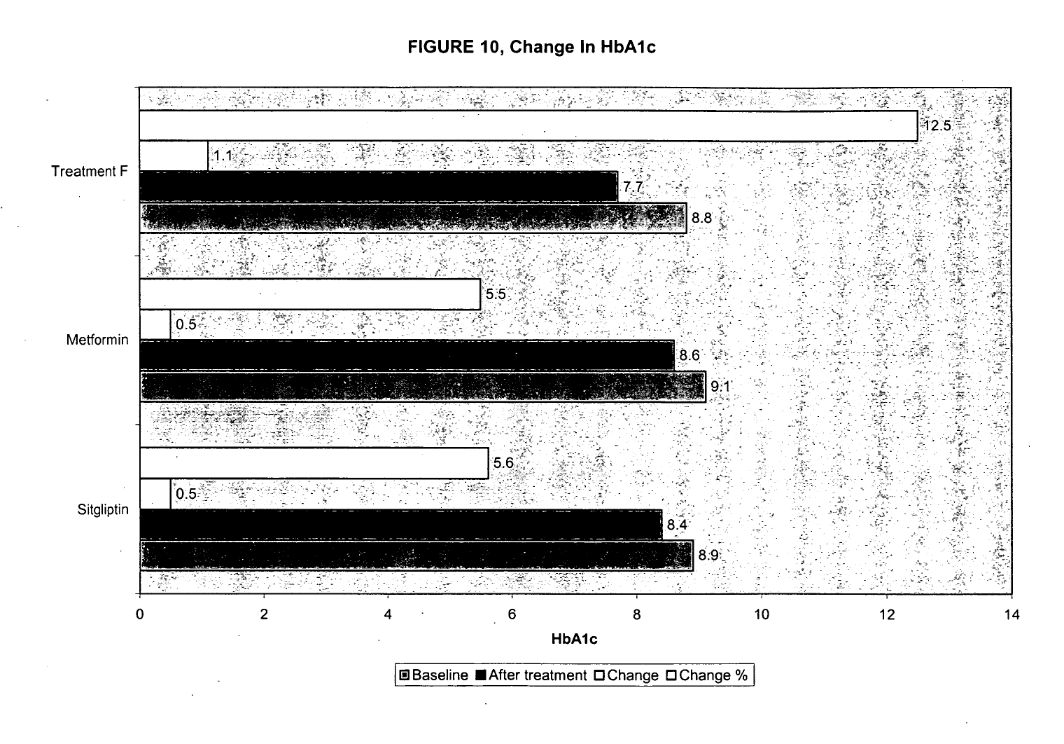

FIG. 10: Change in mean HbA1c for sitagliptin, metformin monotherapy and after three months additional combination therapy using Treatment F comprising sitagliptin phosphate and slow release metformin hydrochloride fixed dose combination.

8. Conclusions

In summary, this invention is related to the novel treatment method of diabetes and diabetes related diseases. The invention provides an anti-diabetic combination comprising a DPP4 inhibitor and a slow release biguanide. For example: The invention was established using a long term human clinical trials with over 100 patients with sitagliptin phosphate and a slow release metformin combination therapy. The combination therapy of DPP4 inhibitor and a slow release biguanide appears to be safe and well-tolerated and can result in significant therapeutic benefits. This combination is exemplified using a combination comprising sitagliptin phosphate (50 mg) and a slow release metformin (500 mg) that was either co-administered or as a fixed dose (Example 1) twice a day to the patients in a long-tem clinical trial.

The foregoing study establishes that the combination of a DPP4 inhibitor and a slow release biguanide causes a clinically significant and unexpected further lowering of fasting glucose compared to either agent used alone. The changes are significant to conclude that the results are due to synergistic effect of a DPP4 inhibitor and the slow release biguanide and rule out the addition effect.

While certain preferred and alternative embodiments of the invention have been set forth for purposes of disclosing the invention, modifications to the disclosed embodiments may occur to those who are skilled in the art. Accordingly, the invention is intended to cover pharmaceutical compositions comprising a slow release biguanide with all DPP4 inhibitors, all embodiments of the invention and modifications thereof which do not depart from the spirit and scope of the invention. Other DPP 4 inhibitors like Vildagliptin and Saxagliptin and any molecule that inhibit the DPP4 protease, including other DPP4 inhibitors referred to as SYR 522, PHX1149, GRC-8200 and SSR-162369, in Recent Patents on Endocrine, Metabolic & Immune Drug Discovery 2007, Vol. 1, No. 1 are incorporated as reference, are encompassed in the scope of this invention².

### REFERENCES

1. Vidon, N., Chaussade, S., Noel, M., Franchisseur, C., Huchet, B., Bernier, J. J. (1988), Diabetes Research and Clinical Practice, 4, 223-239.

2. Recent Patents on Endocrine, Metabolic & Immune Drug Discovery 2007, Vol. 1, No. 1.

3. Pentikainen, P. J. (1986), International Journal of Clinical Pharmacology, Therapy and Toxicology, 24, 213-220.

4. Noel, D. S. (1980), Kinetic study of normal and sustained release dosage forms of metformin in normal subjects, Journal of International Biomedical Information and Data, 1980, pp. 9 to 20.

5. Karttunen et al (1983), The pharmacokinetics of metformin: a comparison of the properties of a rapid-release and a sustained-release preparation, Int. J. Clin. Pharmacology, Therapy and Toxicology, Vol. 21, No. 1, pp. 31-36.

6. FDA labeling information [online], (2006).

7. Raz, I. et al. Efficacy and safety of the dipeptidyl peptidase-4 inhibitor sitagliptin as monotherapy in patients with type 2 diabetes mellitus. Diabetologia 49, 2564-2571 (2006).

8. Aschner, P. et al. Effect of the dipeptidyl peptidase-4 inhibitor sitagliptin as monotherapy on glycemic control in patients with type 2 diabetes. Diabetes Care 29, 2632-2637 (2006).

9. Charbonnel, B. et al. Efficacy and safety of the dipeptidyl peptidase-4 inhibitor sitagliptin added to ongoing metformin therapy in patients with type 2 diabetes inadequately controlled with metformin alone. Diabetes Care 29, 2638-2643 (2006). 10. Rosenstock, J. et al. Efficacy and safety of the dipeptidyl peptidase-4 inhibitor sitagliptin added to ongoing pioglitazone therapy in patients with type 2 diabetes: a 24-week, multicenter, randomized, double-blind, placebo-controlled, parallel-group study. Clin. Ther. 28, 1556-1568 (2006).

11. U.S. Pat. No. 3,845,770,

12. U.S. Pat. No. 3,916,899,

13. U.S. Pat. No. 4,008,719,

14. U.S. Pat. No. 4,034,758,

15. U.S. Pat. No. 4,036,228,

16. U.S. Pat. No. 4,522,625,

17. U.S. Pat. No. 4,612,008

18. U.S. Pat. No. 4,077,407,

19. U.S. Pat. No. 4,783,337,

20. U.S. Pat. No. 5,071,607,

21. U.S. Pat. No. 6,011,049,

22. U.S. Pat. No. 6,099,859,

23. U.S. Pat. No. 6,110,949

24. U.S. Pat. No. 6,153,632,

25. U.S. Pat. No. 6,340,475,

26. U.S. Pat. No. 6,403,121

27. U.S. Pat. No. 6,475,521

28. U.S. Pat. No. 6,451,342,

29. U.S. Pat. No. 6,524,621

30. U.S. Pat. No. 6,676,966

31. WO 01/3594,

32. WO 01/3594,

33. U.S. Pat. No. 7,125,873,

34. WO 9819998,

35. WO 0034241,

1. A method of administering a pharmaceutical composition to a patient in need of such treatment wherein the pharmaceutical composition comprising a therapeutically effective amount of a DPP4 inhibitor and a therapeutically effective amount of a slow release biguanide.
